SEQUENCE LISTING (12 sequences)

SEQ ID NO: 1 - DNA, 18 bp, Artificial Sequence
  Smad4 up primer
gcttcagaaattggagac

SEQ ID NO: 2 - DNA, 19 bp, Artificial Sequence
  Smad4 down primer
cctcagtctaaaggttgtg

SEQ ID NO: 3 - DNA, 61 bp, Artificial Sequence
  Smad3 his up primer
gcgcggccgccatgggcagcagccatcatcatcatcatcacatgtcgtccatcctgccttt

SEQ ID NO: 4 - DNA, 29 bp, Artificial Sequence
  Smad3 his down primer
cgcggatccctaagacacactggaacagc

SEQ ID NO: 5 - DNA, 58 bp, Artificial Sequence
  Smad4 HA up primer
gcgcggccatgtacccatacgacgtcccagactacgctatggacaatatgtctattac

SEQ ID NO: 6 - DNA, 29 bp, Artificial Sequence
  Smad4 HA down primer
cgcggatcctcagtctaaaggttgtgggt

SEQ ID NO: 7 - DNA, 20 bp, Artificial Sequence
  E1A up primer
agctgatcgaagaggtactg

SEQ ID NO: 8 - DNA, 19 bp, Artificial Sequence
  E1A down primer
gagtcacagctatccgtac

SEQ ID NO: 9 - DNA, 22 bp, Artificial Sequence
  E1B up primer
ggttacatctgacctcatggag

SEQ ID NO: 10 - DNA, 22 bp, Artificial Sequence
  E1B down primer
cagtacctcaatctgtatcttc

SEQ ID NO: 11 - DNA, 20 bp, Artificial Sequence
  control up primer
tcgtttctcagcagctgttg

SEQ ID NO: 12 - DNA, 20 bp, Artificial Sequence
  control down primer
catctgaactcaaagcgtgg

(19) United States
(12) Patent Application Publication
(10) Pub. No.: US 2003/0108522 A1
(43) Pub. Date: Jun. 12, 2003

(54) Method for using Smad for gene therapy of solid malignant tumors

(76) Inventors: Je-Ho Lee, Seoul (KR); Seung-Hoon Kim, Seoul (KR)

Correspondence Address: GATES & COOPER LLP, HOWARD HUGHES CENTER, 6701 CENTER DRIVE WEST, SUITE 1050, LOS ANGELES, CA, 90045, US

(21) Appl. No.: 10/231,921

(22) Filed: Aug. 30, 2002

(30) Foreign Application Priority Data
Nov. 15, 2001 (KR) 2001-71120

(51) Int. Cl. A61K 48/00; C12N 15/861; C12N 7/00

(52) U.S. Cl. 424/93.2; 514/44; 435/456; 435/235.1; 435/320.1

(57) ABSTRACT

A method uses Smad for cancer treatment by expressing Smad in solid malignant tumor cells. An adenovirus expression vector includes the Smad4 or Smad3 gene. An adenovirus can produce Smad4 or Smad3 protein within cells by using the above expression vector. A preparation method includes a selection process of RCV-negative clone from adenovirus clones obtained by amplification in packaging cells infected with the adenovirus expression vector and adenovirus mother vector. A method uses the above adenovirus for cancer treatment whereby Smad4 or Smad3 is expressed in solid malignant tumor cells by infecting the cells with adenovirus including Smad. Gene therapy for cancer using Smad treats solid malignant tumors without side effects.

### CROSS REFERENCE TO RELATED APPLICATION

This application claims the benefit of priority to Korean Patent Application No. 2001-71120, filed Nov. 15, 2001, the entire contents of which are incorporated herein by reference.

### FIELD OF THE INVENTION

The present invention relates to a method for using Smad for cancer treatment by expressing Smad in solid malignant tumor cells. More precisely, the present invention relates to an adenovirus expression vector comprising the Smad4 or Smad3 gene, an adenovirus which can produce Smad4 or Smad3 protein within cells by using the above expression vector, a preparation method thereof, and a method for using the above adenovirus for cancer treatment where Smad4 or Smad3 gene is expressed in solid malignant tumor cells by infecting the cells with the adenovirus including Smad.

### BACKGROUND OF THE INVENTION

Gene therapy is a kind of treatment for genetic diseases and cancers caused by an aberration of genes, whose mechanism is to introduce disease-related genes directly to patients in order to normalize the cell function by expressing those genes inside cells. Gene therapy is effective not only for the treatment of diseases, but also for the prevention of diseases and for reinforcing the treatment, since the therapy can bestow a new function on the human body by introducing a specific gene.

The crucial point of gene therapy is how introduced genes can be transferred to the nuclei of target cells successfully for mass expression of the genes. After reaching the target cells, the introduced genes enter the cells through endocytosis and are expressed in nuclei of the cells. DNA genes can be introduced with liposome, a kind of carrier, because DNA itself cannot pass through a cell membrane well. In this case, however, most of the liposome might be destroyed during transfer, resulting in a low transfer efficacy. Using a virus for gene therapy is desirable since foreign genes can be inserted into cells effectively with infectious viruses. Particularly, curable genes should be inserted in virus DNA by a genetic recombination method, and a great amount of these foreign genes are then produced in vitro. By infecting a human body with the virus, the curable genes can be transferred into human cells and expressed effectively. Especially, adenovirus can transfer its gene into the nuclei of cells effectively, which makes it useful for gene therapy.

Transforming growth factor-β (TGF-β) is a kind of growth factor. TGF-β exerts a broad range of biological activities, including cellular proliferation and differentiation. Loss of sensitivity to TGF-β-induced growth inhibition in carcinoma-derived cell lines is a common event and probably an important factor in tumorigenesis (Sporn and Roberts, Cell Regul., 1990, 1:875-882; Dagnino et al., Recent Results Cancer Res., 1993, 128:15-29). Loss of responsiveness to TGF-β growth inhibition has been reported in human cervical tumors (Braun et al., Cancer Res., 1990, 50:7324-7332; Kang et al., Int. J. Cancer, 1998, 77:620-625). The molecular events underlying the loss of TGF-β sensitivity, however, are largely unknown.

Cervical tumorigenesis is a multiple-step process in which infection by human papilloma virus (HPV) has been implicated as an etiologic agent (Park et al., Cancer, 1995, 76:1902-1913). Although HPV-infected keratinocytes occasionally lose sensitivity to TGF-β, it remains unclear whether HPV infection is a direct cause of tumorigenesis (De Geest et al., Gynecol Oncol., 1994, 55:376-385). Loss of the negative growth response to TGF-β in cervical tumorigenesis probably results from the disruption of downstream signaling distal to TGF-β receptor binding. As supporting evidence, changes in receptor numbers and TGF-13 binding affinity do not correlate well with sensitivity to TGF-β1 in squamous carcinoma cell lines (Braun et al., Cancer Res., 1990, 50:7324-7332; Hebert and Birnbaum, Cancer Res., 1989, 49:3196-3202).

Smad proteins play a central role in the TGF-β signaling pathway and constitute a unique framework for signal transduction from the plasma membrane to the nucleus. Specific TGF-β receptor kinases phosphorylate serine residues at the C-terminal ends of Smad1 and Smad5 or Smad2 and Smad3 immediately upon ligand binding (Liu et al., Proc. Natl. Acad. Sci. USA., 1997, 64:10669-74; Souchelnytskyi et al., J. Biol. Chem., 1997, 272:28107-15; Abdollah et al., J. Biol. Chem., 1997, 272:27678-85). Subsequently, these phosphorylated Smads form heterodimers with Smad4, which translocate to the nucleus (Lagna et al., Nature, 1996, 383:832-6), where Smads activate the transcription of target genes (Attisano and Wrana, Curr. Opin. Cell Biol., 2000, 12:235-43; Kijke et al., Trends Biochem Sci., 2000, 25:64-70).

Receptor-specific Smad3 and Smad4 also directly bind DNA since the target motif is present in the promoter of the TGF-β response gene, human PAI-1 (Dennler et al., EMBO J., 1998, 17:3091-100). TGF-β induces transcription of the Smad7 gene, an inducible inhibitor, through activation of Smad3 and Smad4 transcription factors binding to its proximal promoter (von Gersdorff et al., J. Biol. Chem., 2000, 275:11320-6).

Structural and functional studies on the mechanism of Smad4 proteins have provided new insight into the role of genetic mutations in human cancers (Shi et al., Nature, 1997, 388:87-93). For example, mutations in the gene encoding Smads disrupt the essential function of these proteins in TGF-β-induced signaling pathways (Hata et al., Genes Dev., 1998, 12:186-97), and the AML1/Evi-1 chimeric protein interacts with Smad3 through the first zinc finger domain, effectively blocking the growth-inhibitory signal of TGF-β (Kurokawa et al., Nature, 1998, 394:92-6). Although these findings are consistent with Smad proteins possessing an important role in tumor suppression, there remains a need for identifying mechanisms by which Smad proteins might achieve such an effect.

As a result of an analysis of Smad expression that is related to the TGF-β signaling, the present inventors have discovered that TGF-β restrains neither cell growth nor apoptosis in cervical cancer cells and that Smad4 has been specifically less expressed or aberrantly expressed in cervical cancer cells. To express normal Smad protein in cervical cancer cells, the present inventors have prepared an adenovirus expression vector containing the Smad4 or Smad3 gene and then transfected them into packaging cells, from which an adenovirus clone capable of producing Smad4 or Smad3 protein has been selected. The present invention has been accomplished by confirming that growth-inhibiting and apoptotic effects of cervical cancer cells, by TGF-β infected with the above selected adenovirus clone, were largely enhanced and that Smad4 or Smad3 could be effectively used for gene therapy for the treatment of cervical cancer.

### SUMMARY OF THE INVENTION

It is an object of this invention to provide a method for using Smad proteins, such as Smad4 or Smad3, for cancer treatment by expressing Smad4 or Smad3 in solid malignant tumor cells.

To accomplish the object, the present invention provides a method for using Smad4 or Smad3 for cancer treatment by expressing Smad4 or Smad3 in solid malignant tumor cells. The method comprises introducing an exogenous Smad4 or Smad3 gene into solid malignant tumor cells to obtain expression of Smad4 or Smad3 in the tumor cells.

The present invention also provides an adenovirus expression vector containing Smad4 or Smad3 gene.

The present invention also provides an adenovirus that can produce Smad4 or Smad3 proteins within cells by using the above expression vector.

The present invention also provides a preparation method of the above adenovirus.

The present invention also provides a method for using the above adenovirus for the treatment of solid malignant tumors. The invention additionally provides a method for inducing TGF-β1 sensitivity and/or apoptosis in TGF-β1-insensitive cancer cells. The method comprises contacting the TGF-β1-insensitive cancer cells with an expression vector containing an exogenous Smad gene.

In a preferred embodiment, the contacting comprises infecting the cancer cells with an adenovirus modified to produce Smad4 or Smad3 protein. The infecting can be by natural viral entry into the cells or by transfection, as is understood by those skilled in the art.

### BRIEF DESCRIPTION OF THE DRAWINGS

The application of the preferred embodiments of the present invention is best understood with reference to the accompanying drawings, wherein:

FIG. 1 is a chart including a set of photographs showing the results of Northern blot analysis of PAI-1 gene expression by TGF-β1 in cervical cancer cell lines (HeLa, SiHa, Caski, and HT-3);

FIG. 2 is a chart including a set of photographs showing the results of Northern blot analysis of Smad gene expression (Smad1-7) in cervical cancer cell lines;

FIG. 3A is a schematic diagram showing the genetic map of adenovirus expression vector pΔACMVsmad4 containing Smad4 gene;

FIG. 3B is a schematic diagram showing the genetic map of adenovirus expression vector pΔACMVsmad3 containing Smad3 gene;

FIG. 4 is a photograph showing the result of PCR analysis, confirming that the adenovirus clone of the present invention has no replicatin competent recombinant virus (RCV);

FIGS. 5A-5E are charts each including a set of photographs showing the results of Western blot analysis of expression of p21^{Waf1}, phospho-p38, phospho-JNK, phospho-ATF-2, and PAI-1 by TGF-β1, respectively, in cervical cancer cell lines;

FIG. 6 is a graph showing the transcriptional activation of the sTP-lux reporter by TGF-β1 in Smad3- or Smad4-transfected SiHa cells;

FIG. 7A is a chart including a set of photographs showing restored TGF-β1 signaling in SiHa cells by transient transfection of Smad3 and Smad4 expression cDNA. Analysis of apoptosis induced by Smad3 and/or Smad4 transfection in SiHa cells;

FIG. 7B is a photograph showing the SiHa cells transfected with empty vector;

FIG. 7C is a photograph showing the SiHa cells transfected with Smad3/4; and

FIG. 8 is a graph showing the effect of TGF-β1 and Smad3/4 on the growth of a human cervical cell line, SiHa, wherein cell growth rates were determined by thiazolyl blue dye assay.

### DETAILED DESCRIPTION OF PREFERRED EMBODIMENTS

In one aspect, the present invention provides a method for using Smad4 or Smad3 for cancer treatment by expressing Smad4 or Smad3 in solid malignant tumor cells.

TGF-β induces apoptosis and growth-inhibition of cells. When being treated in cervical cancer cells, however, TGF-β cannot affect cell growth or apoptosis since Smad4-encoding protein, which is related to the downstream of TGF-β signaling pathway expressed less or mutated in cervical cancer cells, results in disruption of normal Smad4 function.

In the preferred embodiment of the present invention, the normal Smad4 protein was to be expressed in cervical cancer cells in order to correct the function of mutated Smad4 protein, so that TGF-β can induce cell growth-inhibition and apoptotic cell death even in cervical cancer cells.

The co-expression of Smad4 and Smad3 is preferred to enhance the efficacy of cell growth-inhibition and apoptosis.

The present inventors investigated whether apoptosis was induced when Smad4 or Smad3 was expressed in cervical cancer cells. As a result, apoptosis was induced in cervical cancer cells when Smad4 was expressed therein, even without the TGF-β treatment, and apoptotic cells were remarkably increased when Smad4 and Smad3 were co-expressed.

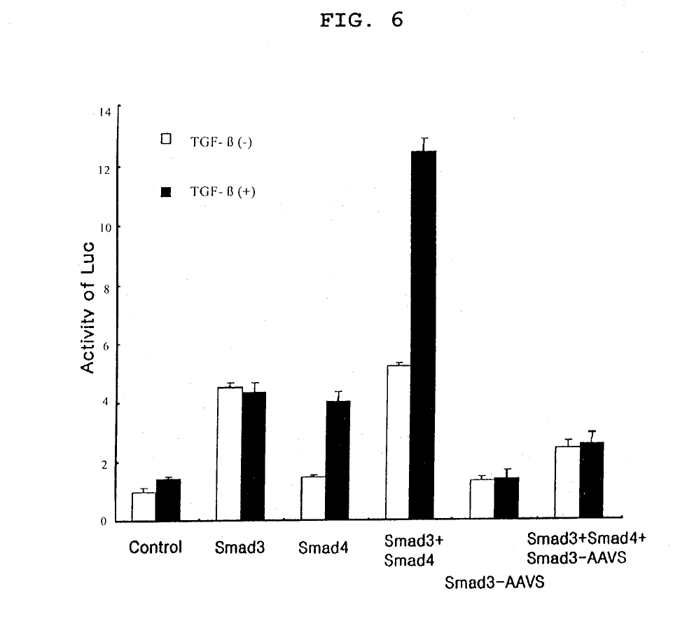

Likewise, cell growth-inhibition and apoptosis were enhanced when Smad4 was expressed in TGF-β1-treated cells. Cell growth-inhibition rate and apoptotic cells were further increased in Smad4 and Smad3 co-expressed TGF-β1-treated cells, over cells that are Smad4 expressed only (see FIGS. 6, 7A-7C, and 8).

Therefore, Smad4 or Smad3 can be widely used for gene therapy for the treatment of solid malignant tumors, since the cell growth-inhibition and apoptosis are induced in Smad4 or Smad3 expressed cells, leading to cell death.

The present invention also provides an adenovirus expression vector containing the Smad4 or Smad3 gene.

The present invention provides an adenovirus expression vector which can produce Smad4 or Smad3 protein by using an expression cassette consisting of coding regions for an immediate early promoter site and multiple cloning site of cytomegalovirus (CMV), and a late polyadenylation signal site of simian virus 40 (SV 40).

As a DNA virus, adenovirus contains E1A gene site essential for virus proliferation in genome and other genes necessary for virus packaging. In order to use adenovirus for gene therapy, genes related to virus proliferation must be removed to avoid causing another disease by self-proliferation and infection in vivo. Thus, eliminating E1A gene site of adenovirus genome, which is related to virus proliferation, results in the safe use of adenovirus for gene therapy, because the virus cannot proliferate itself in normal cells without an E1A gene site.

In the preferred embodiment of the present invention, the E1 gene site was eliminated from the adenovirus genome, and an expression cassette containing Smad4 or Smad3 was inserted into the site. Thus, the expression vector without an E1 gene related to virus proliferation, which can produce Smad4 or Smad3 protein, was constructed.

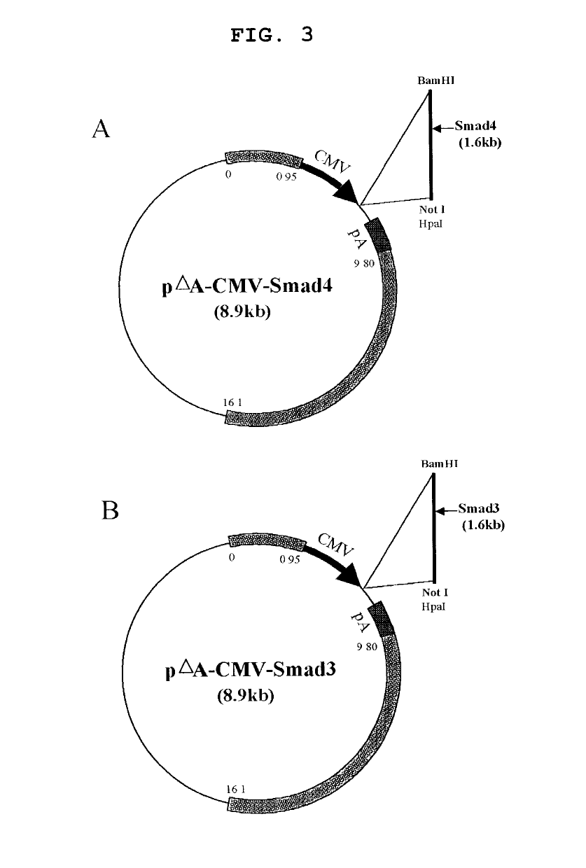

In the present invention, adenovirus expression vector pΔACMVp(A) containing adenovirus type 5 genes (excluding the E1gene) and an expression cassette consisting of coding regions for an immediate early promoter site and multiple cloning site of cytomegalovirus (CMV) and a late polyadenylation signal site of simian virus 40 (SV 40) was used. In order to separate the Smad3 gene, RT-PCR was performed with primers represented by the sequence identification numbers 3 and 4. To separate the Smad4 gene, primers represented by the sequence identification numbers 5 and 6 were used. At this time, RNA purified from normal tissues was used as a template. Finally, normal human Smad3 or Smad4 cDNA was obtained. The above Smad3 or Smad4 cDNA was inserted into a multiple cloning site of pΔACMVp(A) vector, and an expression vector pΔACMVsmad3 or pΔACMVsmad4 was then constructed (see FIG. 3).

The present invention also provides an adenovirus that can produce Smad4 or Smad3 protein within cells by using the above expression vector.

In order to prepare adenovirus massively by using the above adenovirus expression vector, a cell line for adenovirus packaging was transfected with the above expression vector. 293 cells were used for packaging the cell line. The 293 cells contained an E1A gene site of adenovirus in their chromosome DNA, so the E1A gene is expressed continuously within cells and cells are provided with E1A proteins.

The present invention provides an RCV-negative clone selected from adenovirus proliferation by injecting adenovirus expression vector pΔACMVsmad3 or pΔACMVsmad4 into packaging cell line 293 along with adenovirus mother vector.

Adenovirus of the present invention without RCV was named “Ad-CMV-Smad4” or “Ad-CMV-Smad3.” The Ad-CMV-Smad4 was deposited on Nov. 12, 2001 at Gene Bank of Korea Research Institute of Bioscience and Biotechnology (Access No.: KCTC 10115BP).

The present invention provides a preparation method of the above adenovirus.

In order to prepare a great amount of adenovirus using the above adenovirus expression vector, RCV-negative virus clone was selected from adenovirus clones obtained by transfecting adenovirus expression vector into packaging cells along with adenovirus mother vector containing genes essential for adenovirus proliferation.

Due to the great size of adenovirus genome DNA (about 36 kbp), it is difficult to manipulate its genes. Thus, adenovirus expression vector of the present invention includes only parts of the genome rather the whole DNA. The required genes and proteins are provided using adenovirus mother vector. In the preferred embodiments, adenovirus mother vector pJM17 was used to prepare an adenovirus clone, which can produce Smad3 or Smad4 protein in cells.

When adenovirus expression vector was introduced into packaging cells along with adenovirus mother vector, it began to proliferate abundantly and have genes essential for adenovirus packaging expressed. Any cell line that can express E1 protein of adenovirus could be a packaging cell. For the present invention, 293 cells were used as a packaging cell line. Generally, about 10,000 adenovirus particles were produced in a 293 cell. The accumulated virus can be purified easily by crushing and centrifugation.

For the preparation of adenovirus clones useful for gene therapy, it is important to select such adenovirus that does not have RCV. E1 gene site of pΔACMVsmad3 or pΔACMVsmad4, which is the adenovirus expression vector of the present invention, was substituted with the expression cassette expressing Smad3 or Smad4 gene, and the virus produced therefrom could proliferate themselves in none other than 293 cells. However, RCV is frequently generated in the packaging process of 293 cells, even though RCV is not preferable as a clinically used virus since it causes viral infection. Therefore, only RCV-negative virus could be used clinically.

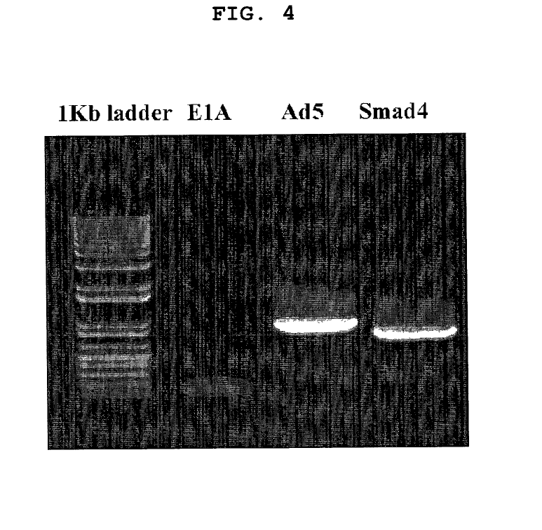

To analyze RCV in an adenovirus clone, PCR was performed since RCV has an El gene site, which the adenovirus expression vector does not have. Particularly, PCR was performed using primers that specifically amplify E1A and E1B genes to confirm whether DNA separated from the above adenovirus clones has E1A and E1B gene sites of adenovirus type 5. 752 bp and 1818 bp DNA fragments could be obtained from the PCR using E1A and E1B primers, when E1 genes exist, from which the existence or absence of RCV can be confirmed (see FIG. 4).

The present invention also provides a method for using the above adenovirus for the treatment of solid malignant tumors. Adenovirus of the present invention can be effectively used for gene therapy of various cancers including cervical cancer.

The present invention also provides a method of gene therapy for cancer using the above adenovirus in which cancer-developed sites are transfected with the adenovirus to inhibit cancer progression or to induce cancer cell death.

In the preferred embodiments of the present invention, cervical cancer cells were infected with the above adenovirus to express normal Smad4 or Smad3 protein, and the infected cells were treated with TGF-β for the cell growth-inhibition and apoptosis induction.

It is preferred to infect cervical cancer cells with adenovirus expressing Smad4 protein. It is more preferable to infect the cells with both adenovirus expressing Smad4 and Smad3.

Adenovirus clones of the present invention can be effectively used for gene therapy of cancer treatment, since adenovirus clones induce cell growth-inhibition and apoptotic cell death of cancer cell lines so infected.

### EXAMPLES

Practical and presently preferred embodiments of the present invention are illustrative as shown in the following Examples. It should be appreciated, however, that those skilled in the art may, on consideration of this disclosure, make modifications and improvements within the spirit and scope of the present invention.

### Example 1

Inhibitory Effect of TGF-β1 on Growth of Cervical Cancer Cells

In order to examine the effect of TGF-β1 on the growth of cervical cancer cells, four established cell lines (HeLa, SiHa, Caski, and HT-3) were used.

<1-1> Cell Culture and Growth-Inhibition Studies

Four human cervical cancer cell lines, i.e., HeLa, SiHa, Caski, and HT-3, were maintained in DMEM supplemented with 10% FCS (Life Technologies, Gaithersburg, Md., USA), amphotericin B (Fungizone, Life Technologies), anti-PPLO (Life Technologies), streptomycin, and penicillin G, at 37° C. in a humidified atmosphere of 5% CO₂.

To examine the effect of TGF-β on cell growth, cervical cancer cells were plated onto 96-well plates (Nalgen Nunc, Naperville, Ill., USA) at densities of 3×10³ cells/well and cultured for one day. After addition of recombinant human TGF-β1 (2 ng/Mg, manufactured by Sigma of St. Louis, Mo.), cell growth was measured with the thiazolyl blue dye assay at the appropriate time points. Particularly, 50 μl of 2 mg/Ml MTT (Boehringer-Mannheim, Mannheim, Germany) were added to each well. Plates were then incubated for three hours at 37° C. After replacing the solutions with 100 μl of DMSO, cell growth was measured by reading the plates at 540nm in an ELISA reader (ThermoMax, Molecular Devices, Menlo Park, Calif., USA).

As a result, HeLa and HT-3 cells were sensitive to TGF-β1, so that their growth was inhibited while SiHa and Caski cells were not. These results are consistent with previous observations that tumorigenic HPV-16⁺ cervical-derived cell lines, SiHa and Caski, are resistant to the growth-inhibitory effect of TGF-β1 (Braun et al., Cancer Res., 1990, 50:7324-7332; Kang et al., Int. J. Cancer, 1998, 77:620-625). TGF-β1 also induced an increase in the invasiveness of Caski, but there was no change in HeLa (Gerard, Int. J. Cancer, 1997, 71:1056-1060). Growth of Caski cells was slightly decreased after the sixth day by TGF-β1 treatment. However, growth HeLa and HT-3 cells was significantly inhibited by TGF-β1.

<1-2> Analysis of PAI-1 mRNA Expression

Because PAI-1 expression is typically regulated by TGF-β1 treatment at the transcriptional level (Keeton et al., J. Biol. Chem., 1991, 266:23048-23052), the present inventors tested the mRNA expression of PAI-1 in order to confirm that TGF-β1 signal pathway in TGF-β1 treated cervical cancer cells worked normally.

Four cervical cancer cell lines were cultured in the presence or absence of TGF-β1 (2 ng/Ml) for six hours. In order to analyze PAI-1 mRNA expression by TGF-β1, Northern hybridization was performed. Particularly, total RNA was isolated by lysing cells in Trizol (manufactured by Gibco BRL of Gaithersburg, Md.) according to the manufacturer's instructions. For Northern blot hybridization, RNA solution (10 μg) was electrophoresed on a 1.2% denaturing agarose gel and transferred onto a nylon membrane (Nytran). ³²P-labeled cDNA probes were synthesized using the Rediprime cDNA synthesis kit (manufactured by Amersham). To normalize hybridized signals, membranes were rehybridized with human glyceraldehydes-3-phosphate dehydrogenase (GAPDH) cDNA. All recombinant DNA used in this study was prepared by RT-PCR amplification and cloning into T-vector (pCR2.1) using the Topo TA Cloning Kit (manufactured by Invitrogen of Carlsbad, Calif.).

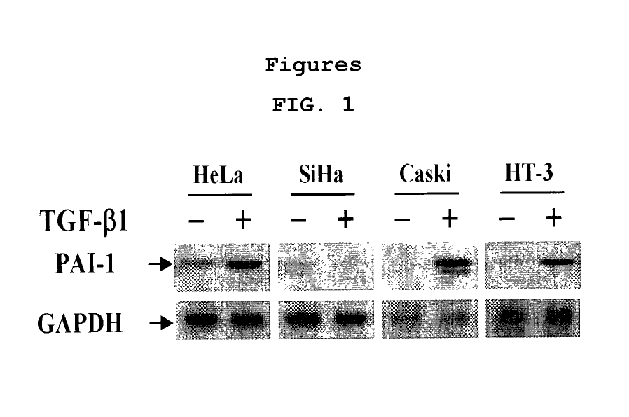

As a result, TGF-β1 remarkably increased PAI-1 mRNA expression in HeLa, Caski, and HT-3 cells, with the induction rate in the Caski cells being the highest. In contrast, TGF-β1 did not increase PAI-1 mRNA expression in SiHa cells (FIG. 1). The failure to induce PAI-1 mRNA expression in SiHa cells indicates that SiHa cells may be defective in TGF-β1 signaling.

### Example 2

Expression of Smads in Cervical Cancer Cells

In order to determine which steps are defective in the TGF-β signaling cascade, the present inventors analyzed the expression of Smad genes (Smads 1-7) by Northern hybridization.

Northern hybridization was performed with ³²P-labeled cDNA probes specific for Smads 1-7. To normalize hybridized signals, the human GAPDH mRNA level was determined. Single membrane was used in all experiments by repeated hybridization and deprobing.

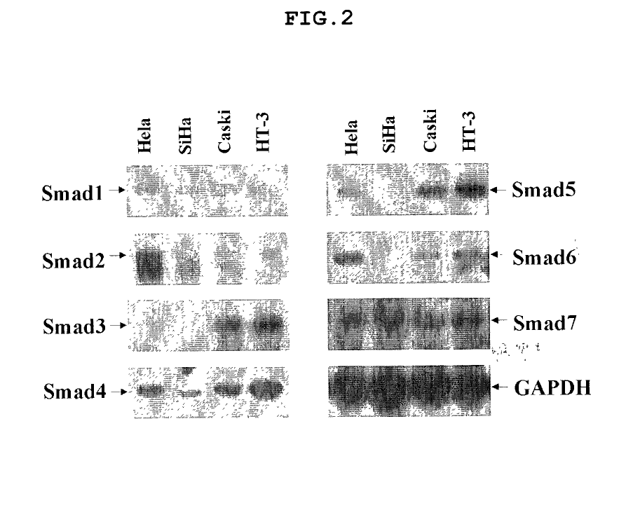

As a result, Smad mRNAs were differentially expressed in the four cervical cancer cell lines (FIG. 2), suggesting that cervical cancer cell lines may use different members of the Smad family in TGF-β1 signaling. The mRNA level of Smad1 was relatively low in all cervical cancer cell lines. In contrast, Smad5 mRNA levels were higher in Caski and HT-3 cells. HeLa cells, however, expressed a much higher level of Smad2 mRNA than the other three cell lines, raising the possibility that TGF-β signaling in HeLa cells might be mediated by Smad2 rather than by Smad3. SiHa cells showed very low expression levels of both Smad2 and Smad3, which are essential for TGF-β-induced signal transduction (Baker and Harland, Curr. Opin. Genet. Dev., 1997, 7:467-473). Smad6 was highly expressed only in HeLa cells. In contrast, Smad7 was expressed at relatively high levels in all four cell lines. Smad4 mRNA signals were detected in all four of the cervical cancer cell lines, but SiHa expressed low levels of Smad4 mRNA compared to the other three lines.

<2-1 > Sequencing Analysis of Smad4 Gene Expressed in Cervical Cancer Cell Lines

The present inventors have sequenced Smad4 gene expressed in cervical cancer cell lines. Particularly, cDNAs of each cell line were used as templates for Smad4 gene amplification, with PCR primers that spanned the entire coding region. PCR was performed with the primers represented by the sequence identification numbers 1 and 2. PCR products were sequenced from both strands by an automated DNA sequencer (“ABI 377,” manufactured by Applied Biosystems of Foster City, Calif.).

As a result, it was confirmed that the Smad4 gene was mutated (Gly²³⁰Ala, Ala⁴⁸⁸Val) in SiHa cells, but the Smad4 was not mutated in the other three cell lines.

<2-2> Secondary Structure of Smad4 Protein Expressed in Cervical Cancer Cell Lines

The present inventors predicted the secondary structural change of the mutated Smad4 by means of the Antheport program (Geourjon et al., J. Mol. Graph., 1991, 9:188-190).

The Ala⁴⁸⁸Val mutation converted the predicted protein structure from a helix to a sheet. These abnormalities of Smad4 in SiHa cells may result in the inactivation of PAI-1 expression and resistance to the growth inhibition exerted by TGF-β1.

### Example 3

Construction of Adenovirus Expression Vector Containing Smad Genes

<3-1> Isolation of Smad3 and Smad4 Genes

In order to isolate Smad3 and Smad4 genes, RT-PCR was performed using Klen Tag polymerase mix (manufactured Clontech of Palo Alto, Calif.). Primers represented by the sequence identification numbers 3 and 4 were used for the isolation of Smad3, and primers represented by the sequence identification numbers 5 and 6 were used for Smad 4. RNA obtained from normal human tissues was used as a template for RT-PCR. PCR primers were designed to include six histidine (His-tag) or hemagglutinin (HA-tag) sequences just before the translation-initiation codons of Smad genes (His-Smad3 and HA-Smad4, respectively). Expression of recombinant Smad proteins was verified by immunoblotting with anti-tag antibodies (“His-probe” and “HA-probe,” manufactured by Santa Cruz Biotechnology of Santa Cruz, Calif.). Verified wild-type Smad3 and Smad4 genes were inserted into pcDNA3.1 expression vectors (manufactured by Invitrogene of the U.S.A.), so that the “pcDNA3.1smad3” and “pcDNA3.1Smad4” were constructed.

A substitutive mutant version of Smad3 was constructed by introducing point mutations by means of the Gene Editor In Vitro Site-Directed Mutagenesis System (manufactured by Promega). The constructed mutant version was named “Smad3-AAVS.”

<3-2> Construction of Adenovirus Expression Vector Containing Smad3 or Smad4

In order to construct adenovirus expression vector containing Smad3 or Smad4 gene, pcDNA3.1smad3 and pcDNA3.1smad4 as prepared in Example <3-1> were digested with BamH I and Not I . The 1.6 kb BamH I , Not I fragment of wild-type human Smad4 or Smad3 was inserted in the multiclonin site of pΔACMVp(A) vector digested with BamH I and Not I . Finally, “pΔACMVsmad3” and “pΔACMVsmad4” were constructed (FIG. 3).

### Example 4

Construction of RCV-Negative Adenovirus Clone which can Produce Smad Proteins within Cells

In order to construct an adenovirus clone which can produce Smad proteins by infecting cells, the expression vector “pΔACMVsmad3” or “pΔACMVsmad4” as prepared in Example <3-2> and an adenovirus mother vector pJM17 (McGrory, et al., Virology, 1988, 163, 614-617) were cotransfected into a packaging cell line, i.e., 293 cells, by a phosphate-calcium method.

The present inventors have confirmed that the separated DNA from the above adenovirus clone has E1A and E1B gene sites of adenovirus type 5 in order to analyze replicatin competent recombinant virus (RCV) residing in adenovirus prepared by using adenovirus expression vector of the present invention. Particularly, PCR was performed with E1A primers represented by the sequence identification numbers 7 and 8 and E1B primers represented by the sequence identification numbers 9 and 10. At this time, adenovirus DNA was isolated by phenol extraction and ethanol precipitation after treating 0.5% SDS containing 2 mg/ml of proteinase K. Through the PCR using E1A and E1B primers, E1 gene site existing in RCV was confirmed by 752 bp and 1818 bp fragments on the agarose gel. When the PCR was performed with E1-unrelated primers represented by the sequence identification numbers 11 and 12, the 816 bp fragment was confirmed regardless of the existence of E1 gene.

In order to determine the number of RCV and to detect the virus proliferation in cells more sensitively, the present inventors used Zhang's method (Zhang, L., et al., Science, 1997, 276, 1268-1272) with slight modification. Virus was subcultured three times in HeLa cells to amplify the RCV Particularly, HeLa cells were infected with a virus clone, and 48 hours after infection, the cells were lysed by a freezing-thawing method. By centrifugation, a supernatant of the cell lysate was obtained. Fresh HeLa cells were infected with the supernatant and then cultured. In order to get virus DNA for PCR, clear cell lysate obtained from subcultured cells was treated with proteinase K, and phenol extraction and ethanol precipitation was performed. The precipitated DNA was dissolved in distilled water, and PCR was performed with E1A primers.

An adenovirus clone without RCV was amplified using 293 cells. Cells were lysed and the lysates were centrifuged with CsCl density gradient. Finally, adenovirus clone for gene therapy was prepared by dialysis with PBS containing 10% glycerol, 1 mM MgCl₂. The number of plaques of 293 cells was measured to determine titer of adenovirus clone of the present invention.

The present inventors named the above-mentioned expression vectors “Ad-CMV-smad4” and “Ad-CMV-Smad3,” and the Ad-CMV-Smad4 of the present invention was deposited on Nov. 12, 2001 at Gene Bank of Korea Research Institute of Bioscience and Biotechnology (Access No.: KCTC 10115BP).

### Example 5

Expression of Proteins Related to Signal Transduction by Smad Protein Expression

The growth-inhibitory effect of TGF-β1 is exerted by arresting cells at the G1 phase and probably by inducing cell death (Masui et al., Proc. Natl. Acad. Sci. USA, 1986, 83:2438-42; Shipley et al., Cancer Res., 1986, 46:2068-71; Lyons and Moses, Eur. J. Biochem., 1990, 187:467-73; Rorker and Jacobberger, Exp. Cell Res., 1995, 216:65-72). Several reports have suggested the involvement of p21^{Waf1}, a cyclin-dependent kinase inhibitor, in TGF-β1-induced G1 arrest (Elbendary et al., Cell Growth Differ. 1994, 5:1301-7; Grau et al., Cancer Res., 1997, 57:3929-34). The present inventors examined whether concomitant induction of p21^{Waf1} occurs following treatment with TGF-β1 in cervical cancer cells. Cells were cultured for six hours in the presence of TGF-β1. The p21^{Waf1} level was determined by Western blot analysis.

Cells were lysed and equal amount of cell extracts were electrophoresed on 15% SDS polyacrylamide gels, electrotransferred onto a nitrocellulose membrane, and probed with antibodies. Smad4, p-p38, p-ATF, JNK1, and pJNK1 were purchased from Santa Cruz Biotechnology. Polyclonal rabbit antihuman p21^{Waf1} antibody and antihuman PAT-1 antibody were obtained from Pharmingen (San Diego, Calif.). Detection was performed using an enhanced chemiluminescence system (“ECL,” manufactured by Amersham of Arlington Heights, Ill.).

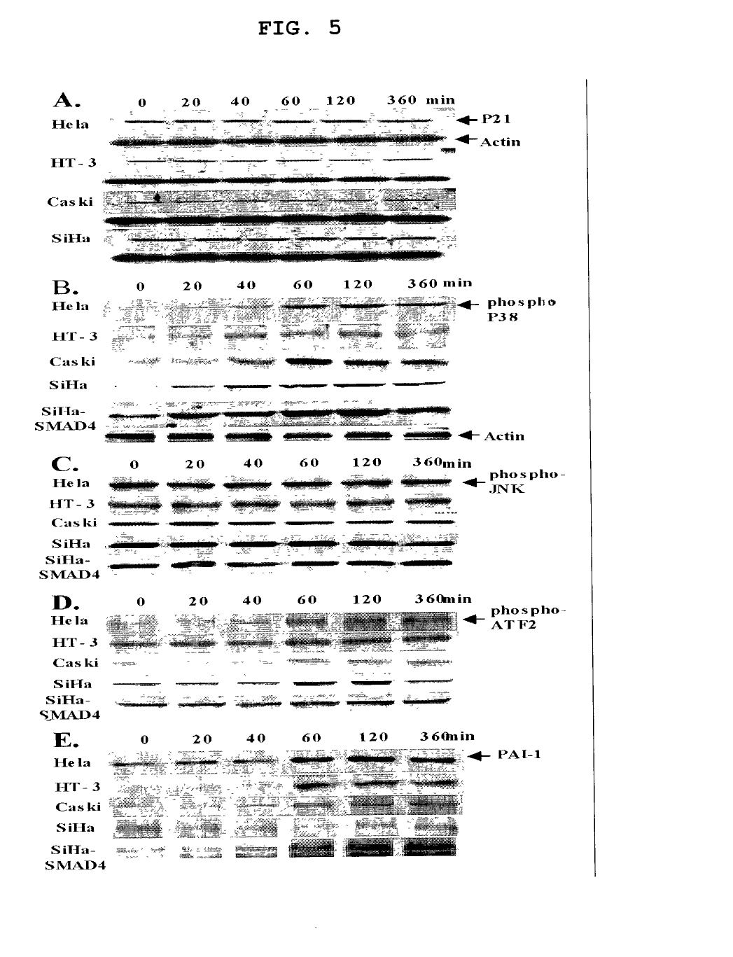

As a result, p21^{Waf1} levels were not changed in any of the cell lines tested (FIG. 5A). Moreover, p21^{Waf1} levels did not show any differences between the control and the TGF-β1-treated cells, even at longer times of the culture period (up to four days). These results indicate that growth inhibition by TGF-β1 may not be mediated by induction of p21^{Waf1} in cervical cancer cell lines.

The fact that p21^{Waf1} levels remained unaltered posed the need for an alternative explanation for the two observations made in SiHa cells treated with TGF-β1, that is, the lack of expression of the PAI-1 gene and the loss of the TGF-β1-induced growth-inhibitory effect. TAK1 acts as a potent activator of the p38 and the SAPK/JNK pathways, and the TAK1 is also required in TGF-β signaling (Sano et al., J. Biol. Chem., 1999, 274:8947-57). Thus, the present inventors investigated both pathways to determine which one is involved with TGF-β1-treated SiHa cells as well as the other cervical cancer cell lines.

The present inventors observed the expression of p38 and ATF2 in Ad-CMV-Smad4-infected SiHa cells and other cervical cancer cell lines (FIG. 5B). The transcription factor ATF-2, phosphorylated by p³⁸ activation, was observed in all TGF-β1-treated cell lines as well as in Ad-CMV-Smad4-infected SiHa cells (FIG. 5D). In contrast, the SAPK/JNK pathway was not activated in any of the TGF-β1-induced cell lines (FIG. 5C).

The present inventors investigated whether the resistant expression of PAI-1 observed in TGF-β1-induced SiHa cells was also found in Ad-CMV-Smad4-infected SiHa cells. PAI-1 expression increased in Ad-CMV-Smad4-infected SiHa cells in a time-dependent manner. In contrast, there were no changes in PAI-1 expression in TGF-β1-treated SiHa cells during the first six hours (FIG. 5E). Therefore, resistant expression of PAI-1 subsequent to TGF-β1 treatment may be caused by mutated and/or scarcely expressed Smad4 in SiHa cells. Moreover, the p38 pathway may also be independent of Smad4 in SiHa cells.

### Example 6

TGF-β1 Sensitivity by Expression of Smad3 or Smad4 in SiHa Cells

SiHa cells were defective in both Smad3 and Smad4 expression, as determined by Northern blot analysis (see FIG. 2). Likewise, response to the TGF-β1 signaling machinery appeared to have been completely lost in these cells. To investigate the correlation between abnormal Smad3/4 expression and loss of TGF-β1 sensitivity, the present inventors studied SiHa cells in complementary experiments that involved transient transfection of Smad 3 and Smad4 assayed six hours after transfection. Particularly, cells were transfected with 3 μg of expression plasmid DNA, 2 μg of reporter, and 1 μg of internal control plasmid using Fugene6 transfection reagent (manufactured by Boehringer-Mannheim) according to the supplier's protocol. The β-galactosidase assay was performed with 1 mg/ml ONPG (Sigma) in a Z buffer as a substrate. 3 TP-Lux reporter cDNA was provided by Dr. P. Chiao (M. D. Anderson Cancer Center, Houston, Tex.) and pcDNA3.1βgal (in vitro gene) was used as an internal control in the transient transfection assay. Luciferase reporter activity was determined in total cell lysates by a luciferase assay system following the manufacturer's instruction (Promega).

As a result, basal activity of the 3 TP-lux reporter gene, which contains PAI-1 promoter, was increased four-fold in Smad3-transfected cells, but there was no increased induction of reporter activity with TGF-β treatment (FIG. 6). This may have been caused by an abnormality of Smad4 in the SiHa cells. Thus, despite the fact that transfection of Smad3 into SiHa cells must have complemented the defective expression of this protein, the increase in Smad3 levels did not cause a further induction of reporter activity in cells treated with TGF-β1.

However, reporter activity was increased in SiHa cells that had been transiently transfected with Smad4. Likewise, the present inventors observed a high basal level of 3 TP-lux reporter gene and a marked increase in reporter activity in cell lines treated with TGF-β1 and cotransfected with both Smad3 and Smad4. These results indicate that insensitivity to the inhibitory effects of TGF-β exhibited by SiHa cells might be due to the fact that Smad4 is a critical cofactor in the TGF-β1 cascade. In other words, aberrant expression of Smad4 and, to a lesser extent, Smad3 may be the cause of loss of responsiveness to TGF-β1 in these cells.

### Example 7

Apoptotic Cell Death by Expression of Smad3 or Smad4

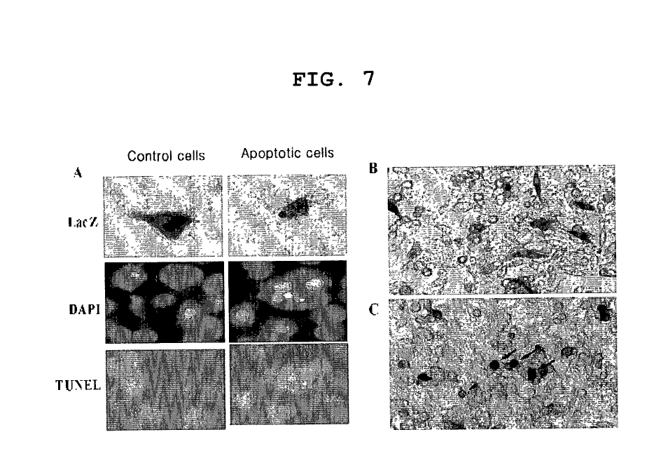

The present inventors performed TUNEL staining on transfected cells to examine whether introduction of Smad3 or Smad4 induces apoptosis of SiHa cells. To discriminate between transfected and non-transfected cells in situ, a β-galactosidase expression vector (pCH110) was cotransfected. After transfected cells were treated for eighteen hours with TGF-β1, TUNEL and X-gal staining were performed. Particularly, 1×10⁵ SiHa cells were plated into a 4-well slide chamber (manufactured by Lab-Tek or Nalgen Nunc) and cultured until 60-70% confluent. Cells were transiently transfected with pCI-neo empty vector or Smad expression vectors together with pCH110 vector. 24 hours after transfection, cells were treated with TGF-β1 (2 ng/ml) for eighteen hours. For in situ TUNEL and LacZ staining, cells were washed three times with ice-cold PBS and fixed with 4% paraformaldehyde (in PBS, pH 7.4). Slides were then treated with permeabilization solution (0.1% Triton X-100 and 0.1% sodium citrate) for two minutes on ice. After several washes with PBS, LacZ staining was performed at 37° C. overnight. The TUNEL assay was performed with the in situ Cell Death Detection Kit (Boehringer-Mannheim). At this time, double-stained cells were considered positive for expression of the introduced molecules and for apoptosis (FIGS. 7A, 7B, and 7C). The percentage of apoptotic cells was determined by dividing the number of TUNEL⁺ cells by the number of X-gal-stained cells (Table 1).

TABLE 1

| | | Percentage of dead cells | |
|---|---|---|---|
| | | Round and | |
| | | detached/ | TUNEL-positive/ |
| | TGF-β1 | LacZ-stained | LacZ- |
| Vectors | (2ng/ml) | cells | stained cells |
| pCI-neo | − | 6.2 (86/1382) | 1.2 (1/82) |
| | + | 8.6 (114/1331) | 3 (1/31) |
| Smad1 | − | 9.3 (66/615) | 2 (2/100) |
| | + | 15.7 (110/656) | 3 (3/100) |
| Smad3 | − | 9.6 (61/637) | 5.4 (2/37) |
| | + | 15.5 (116/750) | 5 (5/100) |
| Smad4 | − | 16.5 (204/1238) | 7 (7/100) |
| | + | 21.7 (199/915) | 13 (13/100) |
| Smad3 + Smad4 | − | 16.9 (208/1230) | 8 (8/100) |
| | + | 19.4 (240/1240) | 20 (20/100) |
| Smad3 + AAVS | − | 9.9 (14/141) | 7 (7/100) |
| | + | 10.5 (15/144) | 10 (10/100) |
| Smad4 + AAVS | − | 6.7 (10/150) | 5 (5/100) |
| | + | 12.0 (18/150) | 10 (10/100) |
| Smad3 + Smad4 + | − | 6.7 (10/150) | 3 (3/100) |
| AAVS | | | |
| | + | 8.7 (13/150) | 4 (4/100) |
| AAVS | − | 3.3 (5/150) | 1 (1/100) |
| | + | 9.3 (14/150) | 2 (2/100) |

As a result, Smad3 alone showed no notable effect on SiHa cell death in the presence or absence of TGF-β1. Smad4, in contrast, increased the number of apoptotic cells even in the absence of TGF-β1 and its effect was further increased by cotransfection with Smad3 (FIGS. 7A, 7B, and 7C). Introduction of Smad3-AAVS, a substitutive mutant of Smad3 (Ser422, 423→Ala), decreased significantly TGF-β1-induced apoptosis of Smad3- and Smad4-transfected SiHa cells, while Smad3-AAVS alone showed no effect.

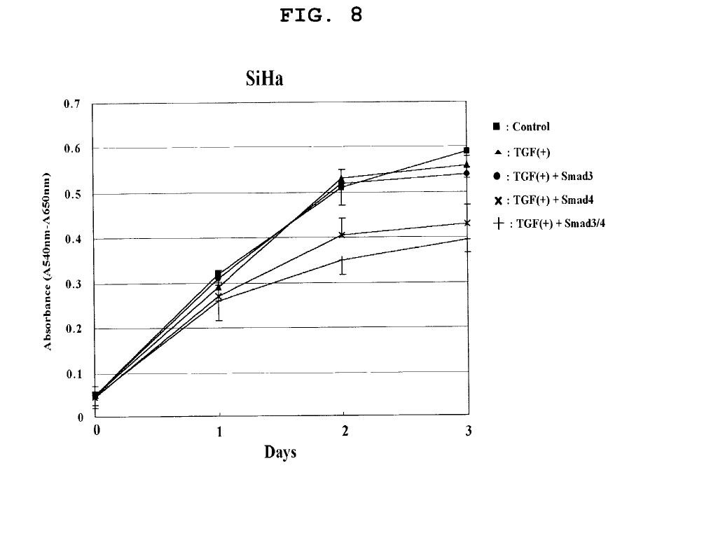

The present inventors also examined the effects of Smad3 and Smad4 on SiHa cell growth by means of the MTT assay. MTT (3-[4,5-Dimethylthiazol-2yl]-2,5-di-phenyltetrazolium bromide) forms blue crystal by reaction with a certain enzyme in mitochondrias of living cells. Survival rate of the cells can be confirmed by the blue colorization. Smad3 or TGF-β1-treated cells showed no growth arrest, but Smad4-treated cells showed a 27% increase in growth arrest. The most notable effect was found in Smad3/Smad4-treated cells, which arrested more than control cells by 33% (FIG. 8).

As shown above, a cancer treatment method of the present invention, whereby Smad3 or Smad4 is expressed in solid malignant tumor cells by infecting adenovirus including Smad3 or Smad4, can be effectively used for gene therapy for the treatment of solid malignant tumors.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

Throughout this application, various references are cited. The entire contents of these references are incorporated herein by reference to describe more fully the state of the art to which the invention pertains.

What is claimed is:

1. A method for treating cancer, the method comprising introducing an exogenous Smad gene into solid malignant tumor cells.

2. The method as set forth in claim 1, wherein the Smad is Smad3.

3. The method as set forth in claim 1, wherein the Smad is Smad4.

4. The method as set forth in claim 1, wherein the solid malignant tumor is cervical cancer.

5. The method as set forth in claim 1, further comprising introducing exogenous TGF-β into the solid malignant tumor cells.

6. The method as set forth in claim 1, wherein the exogenous Smad gene is introduced into the solid malignant tumor cells by infecting the solid malignant tumor cells with an adenovirus containing the exogenous Smad gene.

7. An adenovirus expression vector containing a Smad gene.

8. The adenovirus expression vector as set forth in claim 7, wherein the Smad gene comprises a Smad3 gene.

9. The adenovirus expression vector as set forth in claim 7, wherein the Smad gene comprises a Smad4 gene.

10. The adenovirus expression vector as set forth in claim 7, wherein the vector lacks a functional E1 gene.

11. The adenovirus expression vector as set forth in claim 7, wherein the vector contains an immediate promoter site and multiple cloning site of cytomegalovirus (CMV), a late polyadenylation signal site of simian virus 40, and a Smad4 gene.

12. The adenovirus expression vector as set forth in claim 7, wherein the expression vector is pΔACMVsmad4.

13. The adenovirus expression vector as set forth in claim 7, wherein the vector contains an immediate promoter site and multiple cloning site of cytomegalovirus (CMV), a late polyadenylation signal site of simian virus 40, and Smad3 gene.

14. The adenovirus expression vector as set forth in claim 13, wherein the expression vector is pΔACMVsmad3.

15. An adenovirus constructed by amplification in packaging cells infected with the adenovirus expression vector of claim 7.

16. The adenovirus as set forth in claim 15, wherein the packaging cells can produce E1 proteins.

17. The adenovirus as set forth in claim 16, wherein the packaging cells are 293 cells.

18. The adenovirus as set forth in claim 15, wherein the adenovirus is Ad-CMV-Smad4 (Accession No.: KCTC 10115BP).

19. The adenovirus as set forth in claim 15, wherein the adenovirus is Ad-CMV-Smad3.

20. A method of preparing an adenovirus expression vector that contains an immediate promoter site and multiple cloning site of cytomegalovirus (CMV), a late polyadenylation signal site of simian virus 40, and a Smad4 gene, wherein the method comprises selecting an RCV-negative clone from adenovirus clones obtained by amplification in packaging cells infected with the adenovirus expression vector of claim 7 and an adenovirus mother vector.

21. A method for treating solid malignant tumors comprising contacting a solid malignant tumor with the adenovirus of claim 15.

22. The method as set forth in claim 21, wherein the solid malignant tumor is cervical cancer.

23. The method as set forth in claim 22, wherein the adenovirus is Ad-CMV-Smad4 (Accession No.: KCTC 10115BP).

24. The method as set forth in claim 23, further comprising contacting the solid malignant tumor with adenovirus Ad-CMV-Smad3.

25. The method as set forth in claim 21, further comprising contacting the solid malignant tumor with TGF-β.
